(19) United States
(12) Patent Application Publication
(10) Pub. No.: US 2026/0007387 A1
(43) Pub. Date: Jan. 8, 2026

(54) ULTRASOUND DIAGNOSIS SYSTEM, AND FORCE MEASUREMENT METHOD THEREOF

(71) Applicant: AIRS MEDICAL INC., Seoul (KR)

(72) Inventor: Changkyun KIM, Seoul (KR)

(21) Appl. No.: 19/135,954

(22) PCT Filed: Nov. 10, 2023
(86) PCT No.: PCT/KR2023/018088

§ 371 Date: Sep. 24, 2025

(30) Foreign Application Priority Data
Dec. 7, 2022 (KR) 10-2022-0169967

(51) Int. Cl. A61B 8/00

(52) U.S. Cl. CPC A61B 8/429; A61B 8/4254; A61B 8/4281; A61B 8/54

(57) ABSTRACT

The present invention is directed to an ultrasound diagnosis system and a force measurement method therefor. The ultrasound diagnosis system may include: a probe module including: a force sensor configured to provide force data obtained by measuring the force applied to an affected part; an attitude measurement sensor configured to provide the angle data obtained by measuring the angle formed between the height and vertical directions of an ultrasound probe; and a gel pad provided at an end of the ultrasound probe; and a control unit configured to calculate the first contact force applied by the ultrasound probe to the affected part by using an ultrasound image of the affected part, the force data, and the angle data, and to calculate the second contact force applied by the ultrasound probe to a target area of the affected part to be observed.

### CROSS-REFERENCE TO RELATED APPLICATION

This patent application is a national stage application of PCT/KR2023/018088 filed on Nov. 10, 2023, which claims the priority and benefits of Korean patent application No. 10-2022-0169967, filed on Dec. 7, 2022, the disclosure of which is incorporated herein by reference in its entirety.

### TECHNICAL FIELD

The present disclosure relates to an ultrasound diagnosis system and force measurement method therefor that are capable of determining the force that is applied by an ultrasound probe to an affected part.

### BACKGROUND ART

An ultrasound diagnosis system is a system that acquires an ultrasound image of the tissue inside an affected part by transmitting ultrasonic waves into a living body via an ultrasound probe and then receiving ultrasonic waves reflected from boundaries between tissues with different acoustic impedances via the ultrasound probe.

The ultrasound diagnosis system includes an ultrasound probe, and a probe module configured to generate an ultrasound image of the inside of an affected part by using an ultrasound image acquired via the ultrasound probe. The probe module may output an ultrasound image onto the display unit of the ultrasound diagnosis system, and a diagnostician may diagnose the affected part via the ultrasound image output onto the display unit.

This ultrasound diagnosis system may be used for a specific medical examination such as biopsy. Biopsy is a method of extracting a portion of tissue from a living body and examining it. According to the biopsy, while operating the ultrasound diagnosis system, a diagnostician brings the ultrasound probe into contact with the surface of a living body and inserts a needle into the living body (in-vivo). In this case, the diagnostician may simultaneously check an image of the tissue in the living body and an image of the movement of the needle on the display unit of the ultrasound diagnosis system, may insert the needle into a target part for biopsy, and then extract tissue from the target part. Meanwhile, a needle used to inject a drug into a target part may also be inserted into the target part in the above-described manner.

When an operator such as a doctor or nurse performs manual ultrasound imaging by using the ultrasound diagnosis system, the operator performs imaging while manually controlling the force with which the ultrasound probe is pressed by hand, so that an ultrasound image can be intuitively matched with the force with which the ultrasound probe is pressed onto an affected part. The ultrasound images acquired will vary depending on the force (e.g., 1N, 5N, or 10N) with which the operator presses the ultrasound probe.

However, in the case of an ultrasound diagnosis system that adjusts the position and angle of an ultrasound probe based on robotic guide technology, when there is no sensor unit that measures the force with which the ultrasound probe presses an affected part, there is a problem in that it may be impossible to know the force with which the ultrasound probe presses the affected part, and thus, it may be impossible to determine the degree of pressing force with which an ultrasound image was captured. Accordingly, the ultrasound diagnosis system provides data on the force applied by the ultrasound probe to an affected part together with an ultrasound image.

Since the degree of deformation of a target part (e.g., a blood vessel, or the like) within an affected part varies depending on the force with which the ultrasound probe presses the affected part, the ultrasound diagnosis system acquires an ultrasound image reflecting the target part deformed based on the force with which the ultrasound probe presses the affected part. A diagnostician monitors the blood vessel as the target part in the ultrasound image and determines the suitability for a medical procedure such as blood collection, intravenous (IV) injection, or the like.

Conventional ultrasound diagnosis systems only provide the total force applied by an ultrasound probe to an overall affected part as force data, and may not provide data on the local force applied to a target part such as a vein, an artery, or a nerve. A diagnostician may not determine whether the shape of a blood vessel is originally oval or is a shape pressed by an ultrasound probe by using only an ultrasound image of an oval-shaped blood vessel and data on the force applied by the ultrasound probe to an affected part. As a result, the diagnostician may not accurately evaluate the shape of the blood vessel, making it difficult to determine the precise location or suitability of a medical procedure.

### DISCLOSURE

### Technical Problem

The present disclosure has been conceived in response to the above-described background art, and an ultrasound diagnosis system and a force measurement method therefor according to one embodiment of the present disclosure are intended to, during ultrasound imaging, measure the force applied by an ultrasound probe to an affected part and the force applied by the ultrasound probe to a target part of the affected part to be observed.

However, the objects to be achieved in the present disclosure are not limited to the object mentioned above, and other objects not mentioned may be clearly understood based on the following description.

### Technical Solution

There are disclosed an ultrasound diagnosis system and a force measurement method therefor according to embodiments of the present disclosure for achieving the above-described object. The ultrasound diagnosis system may include: a probe module including: a force sensor configured to provide force data obtained by measuring the force applied by an ultrasound probe to an affected part; an attitude measurement sensor configured to provide the angle data obtained by measuring the angle formed between the height and vertical directions of the ultrasound probe; and a gel pad provided at an end of the ultrasound probe and configured to come into contact with the affected part; and a control unit configured to calculate the first contact force applied by the ultrasound probe to the affected part by using an ultrasound image of the affected part acquired via the probe module, the force data, and the angle data, and to calculate the second contact force applied by the ultrasound probe to a target area of the affected part to be observed based on the first contact force.

Alternatively, the gel pad may include an elastic member whose shape is deformed in accordance with the curvature of the surface of the affected part.

Alternatively, the first contact force may be calculated by compensating for the force applied to the affected part by the mass of the ultrasound probe based on the force data.

Alternatively, the first contact force is calculated by adding the force data to a value determined by the mass of the ultrasound probe and the angle data.

Alternatively, the ultrasound image may reflect therein a deformed state that occurs as the gel pad comes into contact with the affected part; and the control unit may extract starting and ending points at which the gel pad comes into contact with the affected part from the ultrasound image and calculate a first contact area in which the gel pad comes into contact with the affected part, and may extract the starting and ending points of the target part and calculate a second contact area in which the gel pad comes into contact with the target part.

Alternatively, the second contact force may be calculated as the force applied to the second contact area based on the first contact force and the first contact area.

Alternatively, the second contact force may be calculated using the first contact force, the thickness deformation amount of a local area for the target part, and the thickness deformation amount of an overall area in which the gel pad comes into contact with the affected part.

Alternatively, the second contact force may be calculated based on Equation 1 by considering the thickness of the gel pad and the thickness of the gel pad corresponding to any x coordinate. Meanwhile, there is disclosed a force measurement method for an ultrasound diagnosis system according to embodiments of the present disclosure for achieving the above-described object. The force measurement method is performed by an ultrasound diagnosis system including an ultrasound probe, and the force measurement method may include: as an ultrasound image of an affected part is acquired via an ultrasound probe, measuring the force data applied by the ultrasound probe to the affected part and the angle data formed by the height and vertical directions of the ultrasound probe; and calculating the first contact force applied by the ultrasound probe to the affected part and the second contact force applied by the ultrasound probe to a target area of the affected part to be observed by using the ultrasound image, the force data, and the angle data.

Alternatively, calculating the first contact force may further include calculating the first contact force by compensating for the force with which the affected part is pressed by the mass of the ultrasound probe based on the force data.

Alternatively, calculating the first contact force may further include calculating the first contact force by using the force data measured by a force sensor of the ultrasound probe, the angle data measured by an attitude measurement sensor of the ultrasound probe, the mass of the ultrasound probe, and gravitational acceleration.

Alternatively, calculating the first contact force may include calculating the first contact force by compensating the force data for the value determined by the mass of the ultrasound probe and the angle data.

Alternatively, calculating the second contact force may further include: extracting starting and ending points, at which a gel pad comes into contact with the affected part, from the ultrasound image reflecting therein a deformed state that occurs as the gel pad provided at an end of the ultrasound probe comes first into contact with the affected part, and calculating a f contact area in which the gel pad comes into contact with the affected part; and extracting starting and ending points of the target part from the ultrasound image, and calculating a second contact area in which the gel pad comes into contact with the target part.

Alternatively, calculating the second contact force may further include calculating the second contact force applied to the second contact area based on the first contact force and the first contact area.

Alternatively, calculating the second contact force may include calculating the second contact force by using the first contact force, the thickness deformation amount of a local area for the target part, and the thickness deformation amount of an overall area in which the gel pad comes into contact with the affected part.

Alternatively, calculating the second contact force may further include calculating the second contact force based on Equation 3 below by considering the thickness of the gel pad provided at the end of the ultrasound probe and configured to come into contact with the affected part and the thickness of the gel pad corresponding to any x coordinate.

### Advantageous Effects

The ultrasound diagnosis system and the force measurement method therefor according to one embodiment of the present disclosure may know not only the global force applied by the ultrasound probe to an affected part but also the local force applied by the ultrasound probe to a target part of the affected part to be observed during imaging, ultrasound so that considerably precise reading via an ultrasound image is enabled.

In addition, the ultrasound diagnosis system and the force measurement method therefor according to one embodiment of the present disclosure may extract the boundary line within which the gel pad comes into contact with an affected part in an ultrasound image by performing an edge detection algorithm in a computing environment utilizing the characteristic in which the gel pad provided at an end of the ultrasound probe is deformed in accordance with the curvature of the surface of an affected part, so that the contact area where the gel pad comes into contact with the affected part can be known, so that there is no need to add more sensors, and thus, there is an advantage in that no additional cost is incurred.

### DESCRIPTION OF DRAWINGS

FIG. 1 is a block diagram illustrating the configuration of an ultrasound diagnosis system according to one embodiment of the present disclosure;

FIG. 2A to 2D show diagrams illustrating the configuration of a probe module according to one embodiment of the present disclosure;

FIG. 3 is an exemplary diagram illustrating the force applied to a probe module according to one embodiment of the present disclosure;

FIG. 4 is a diagram illustrating the configuration of a gel pad according to one embodiment of the present disclosure;

FIGS. 5A and 5B are exemplary diagrams illustrating an ultrasound image acquired by an ultrasound probe according to one embodiment of the present disclosure; and

FIG. 6 is a flowchart illustrating a force measurement method for an ultrasound diagnosis system according to one embodiment of the present invention.

### MODE FOR INVENTION

Embodiments of the present disclosure will be described in detail below with reference to the accompanying drawings so that those having ordinary skill in the art of the present disclosure (hereinafter, those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “at least one of A and B” used herein should be interpreted to refer to all of A, B, and the combination of A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “N-th (N is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “connected” used herein should be interpreted to include not only a case where components are “directly connected” to each other but also a case where another component is “present” between components and a case where components are “electrically connected” with another component interposed therebetween.

The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

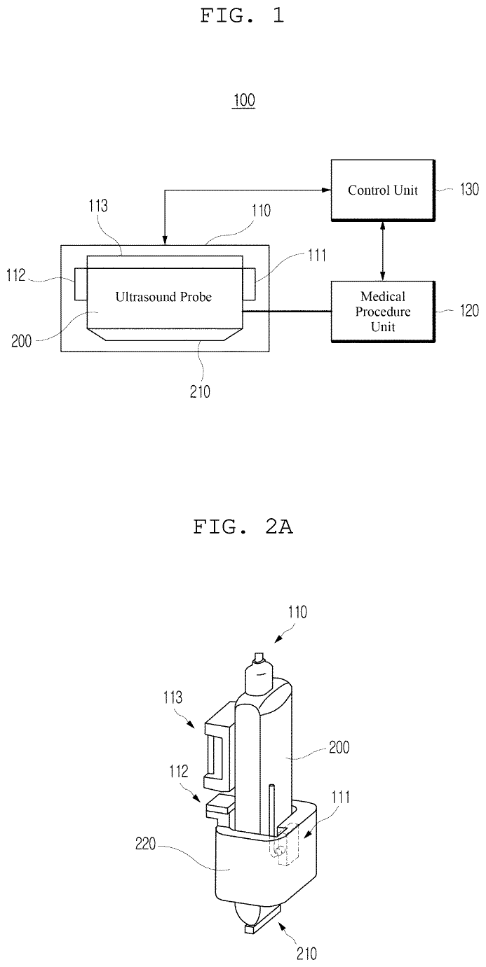

FIG. 1 is a block diagram illustrating the configuration of an ultrasound diagnosis system according to one embodiment of the present disclosure.

Referring to FIG. 1, an ultrasound diagnosis system 100 includes, but is not limited to, a probe module 110, a medical procedure unit 120, and a control unit 130.

The probe module 110 includes a force sensor 111, an attitude measurement sensor 112, an interface part 113, and an ultrasound probe 200. In this case, the probe module 110 may include a distance sensor and at least one infrared camera, and may provide position or coordinate information in order to control the movement of the ultrasound probe 200 and the medical procedure unit 120 during automatic blood collection.

The force sensor 111 measures the force applied to an affected part by the ultrasound probe 200 and provides force data, and the attitude measurement sensor 112 measures the angle formed between the height and vertical directions of the ultrasound probe 200 and provides angle data. The attitude measurement sensor 112 is an Attitude Heading Reference System (AHRS) sensor, includes an acceleration sensor, a gyro sensor, and a geomagnetic sensor in a single sensor, and may measure the acceleration, angular velocity, and geomagnetism of the sensor in a three-dimensional space and provide sensor values in the form of vector data. Furthermore, the AHRS sensor may communicate through the I2C communication protocol and includes a power regulator therein, so that it can use low power (3.3 V to 5 V).

The interface part 113 performs an interface function between the ultrasound probe 200 and the control unit 130, may transmit and receive electrical signals to and from the control unit 130, and may also receive a control signal intended to control each component of the ultrasound probe 200. For example, the interface part 113 may be a connector. When the ultrasound probe 200 is connected to the control unit 130 through a wired connection, the interface part 113 of the ultrasound probe 200 may be connected to the control unit 130 via a cable.

The ultrasound probe 200 transmits ultrasonic waves to an affected part, receives ultrasonic echo signals from the affected part, converts the ultrasonic echo signals into electrical signals, and transmits the electrical signals to the control unit 130.

The ultrasound diagnosis system 100 may perform a medical procedure such as blood collection by using the medical procedure unit 120 including a needle mounting part (not shown) and a needle (not shown).

The control unit 130 may include an input/output unit, a processor, memory, a display unit, and a network unit. The control unit 130 may include other components for implementing a computing environment. Furthermore, only some of the components disclosed above may be included in the control unit 130.

The control unit 130 may control the overall operation of the ultrasound diagnosis system 100. The control unit 130 may generate control commands based on user commands via the input/output unit, and may control the components of the ultrasound diagnosis system 100. Alternatively, the control unit 130 may generate an ultrasound image of an affected part based on an ultrasonic signal received from the ultrasound probe 200, and may display it on the display unit. The control unit 130 may be connected to the ultrasound probe 200 via a wired or wireless connection.

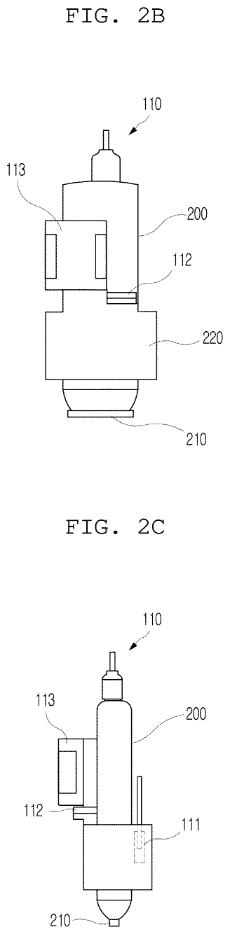

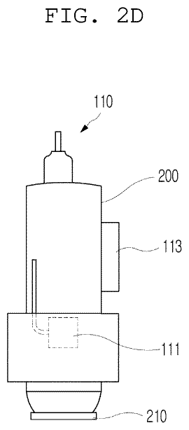

FIG. 2A to 2D show diagrams illustrating the configuration of a probe module according to one embodiment of the present disclosure, and FIG. 3 is an exemplary diagram illustrating the force applied to a probe module according to one embodiment of the present disclosure.

Referring to FIG. 2A to 2D, the probe module 110 may include a gel pad 210 configured to be disposed at an end of the ultrasound probe 200 and come into contact with an affected part, and the interface part 113 installed on one side surface of the upper end portion thereof.

The probe module 110 includes a connection part 220 between the gel pad 210 and the interface part 113, the force sensor 111 may be built into one side of the connection part 220, and the attitude measurement sensor 112 may be installed on the other side of the connection part 220. The inside of the connection part 220 may include a connection wire that is electrically connected to the force sensor 111 and the attitude measurement sensor 112 and that may transmit the data measured by the force sensor 111 and the attitude measurement sensor 112. This connection part 220 may be formed in a hollow frame shape and fitted over the outer surface of the lower end of the ultrasound probe 200.

The connection part 220 is disposed on the hand grip portion of the ultrasound probe 200 and may have a connection wire formed therein, so that wiring design or arrangement for connecting the individual sensors is facilitated and also the sterilization of the ultrasound probe 200 or gel pad 210 may be facilitated.

In this case, the force sensor 111 and the attitude measurement sensor 112 are installed to be positioned in the opposite directions of the connection part 220. For example, in the case where the an operator brings the ultrasound probe 200 into contact with an affected part and moves the ultrasound probe 200, when the attitude measurement sensor 112 is positioned above the connection part 220 based on the affected part, the force sensor 111 may be positioned in the lower portion of the connection part 220, as shown in FIG. 3.

Meanwhile, pressure generally refers to the force applied per unit area. That is, when the unit area is known, force may be obtained via pressure, and pressure may be obtained via force. Accordingly, in this specification, the force between the ultrasound probe 200 and an affected part may refer contact force or contact pressure. More specifically, a first contact force for a first contact area may be the global force data that is applied to an affected part by the ultrasound probe 200, and a second contact force calculated using a second contact area may be the local force data that is applied to a target part by the ultrasound probe 200.

The force data Fₛₑₙₛₒᵣ measured by the force sensor 111 may be represented by Equation 1 below:

$${F}_{sensor} = {F}_{probe} - {m}_{probe} g cos θ \qquad ( 1 )$$

F_{probe} denotes the first force applied by the ultrasound probe 200 to an affected part, m_{probe} denotes the mass of the ultrasound probe 200, θ denotes the angle data obtained via the attitude measurement sensor 112 and formed by the height and vertical directions of the ultrasound probe 200, and g denotes the gravitational acceleration.

Accordingly, the first contact force F_{probe} may be calculated by adding the force data to the value determined by the mass of the ultrasound probe and the angle data. More specifically, the first contact force F_{probe} may be represented by Equation 2 below by compensating for the force with which the affected part is pressed by the mass of the ultrasound probe 200 by using the attitude measurement sensor 112.

$${F}_{probe} = {F}_{sensor} + {m}_{probe} g cos θ \qquad ( 2 )$$

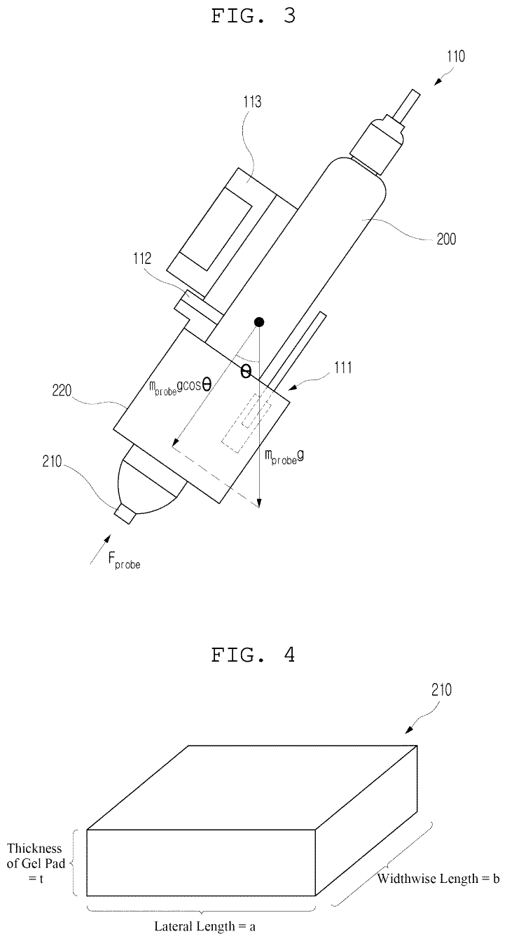

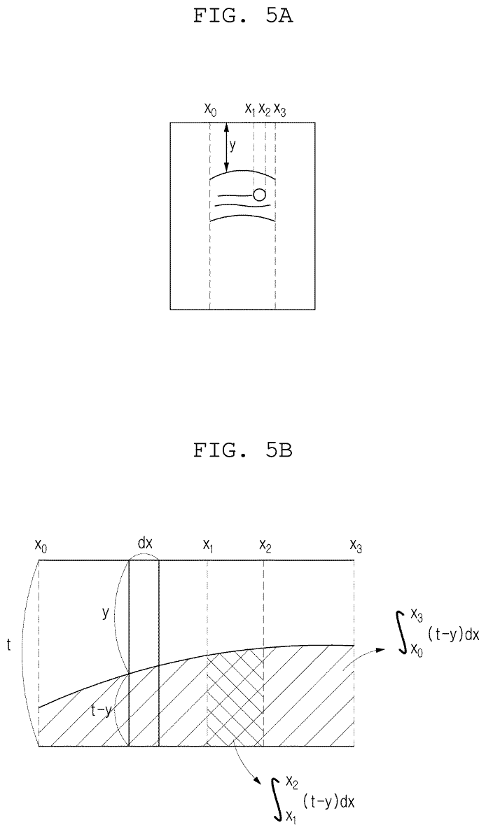

FIG. 4 is a diagram illustrating the configuration of a gel pad according to one embodiment of the present disclosure, and FIGS. 5A and 5B are exemplary diagrams illustrating an ultrasound image acquired by an ultrasound probe according to one embodiment of the present disclosure.

As shown in FIGS. 4, 5A and 5B, the gel pad 210 used for ultrasound imaging may be made of an elastic material such as silicone. When the gel pad 210 comes into contact with the curved surface of an affected part due to the elastic material, the gel pad 210 is deformed into a shape corresponding to the curvature of the surface of the affected part. Accordingly, the ultrasound image reflects therein the contact portion where the gel pad 210 comes into contact with the affected part and the portion where it does not come into contact with the affected part due to the characteristics of the gel pad 210.

When the ultrasound image reflecting therein the deformation state that occurs as the gel pad 210 comes into contact with the affected part is examined, it can be seen that the deformation occurs more in the area of the gel pad 210 which comes into contact with the affected part and in which the pressing force is the maximum and the deformation occurs less as the pressing force decreases.

Using this, the second contact force F_{target} that is applied by the ultrasound probe 200 to the target part of the affected part to be observed may be calculated.

The second contact force F_{target} may be obtained by integrating the strain for a local area in the overall area where the deformation of the gel pad 210 has occurred based on the first contact force F_{probe} and the ultrasound image to thus obtain the force applied to the target part. In this case, the local area may refer to the area corresponding to the target part. That is, the second contact force may be calculated using the first contact force, the thickness deformation amount of the local area for the target part, and the thickness deformation amount of the overall area in which the gel pad comes into contact with the affected part.

More specifically, the second contact force F_{target} may be obtained based on Equation 3 below. Equation 3 below is based on the assumption that the gel pad 210 satisfies Hooke's law σ=Ex. In Hooke's law, E denotes the elastic coefficient, σ denotes the deformation force/stress, and ε denotes the strain. Meanwhile, when the gel pad 210 approximately follows Hooke's law, Equation 3 may be established within an approximate range. Furthermore, Equation 3 may be modified and applied within a similar range depending on the physical characteristics of the gel pad 210 and the environment surrounding the gel pad 210.

$$\begin{matrix}{F}_{target} = {F}_{probe} \frac{{F}_{target}}{{F}_{probe}} \\ = {F}_{probe} \frac{b {\int }_{{x}_{1}}^{{x}_{2}} σ dx}{b {\int }_{{x}_{0}}^{{x}_{3}} σ dx} \\ = {F}_{probe} \frac{bE {\int }_{{x}_{1}}^{{x}_{2}} ε dx}{bE {\int }_{{x}_{0}}^{{x}_{3}} ε dx} \\ = {F}_{probe} \frac{bE {\int }_{{x}_{1}}^{{x}_{2}} ( 1 - \frac{y}{t} ) dx}{bE {\int }_{{x}_{0}}^{{x}_{3}} ( 1 - \frac{y}{t} ) dx} \\ = {F}_{probe} \frac{{\int }_{{x}_{1}}^{{x}_{2}} ( t - y ) dx}{{\int }_{{x}_{0}}^{{x}_{3}} ( t - y ) dx}\end{matrix} \qquad ( 3 )$$

In Equation 3, t denotes the thickness of the gel pad 210, y denotes the thickness of the gel pad 210 at a corresponding x coordinate, x₀ denotes the x-coordinate starting point of the contact portion of the gel pad 210 in the ultrasound image, x₁ denotes the x-coordinate starting point of the target part in the ultrasound image, x₂ denotes the x-coordinate ending point of the target part in the ultrasound image, and x₃ is the x-coordinate ending point of the contact portion of the gel pad 210 in the ultrasound image.

The control unit 130 may extract the starting point x₀ and ending point x₃ of the contact portion of the gel pad 210 and the starting point x₁ and ending point x₂ of the target part by using an edge detection algorithm. The control unit 130 may calculate the first contact area, which is the area where the gel pad 210 comes into contact with the target part, by using x₀ and x₃, and may calculate the second contact area, in which the gel pad 210 comes into contact with the target part of the first contact area, by using x₁ and x₂.

That is, via the present invention, the second contact force applied to the target area may be accurately determined without considering the mechanical characteristics of the gel pad 210, which is an elastic member.

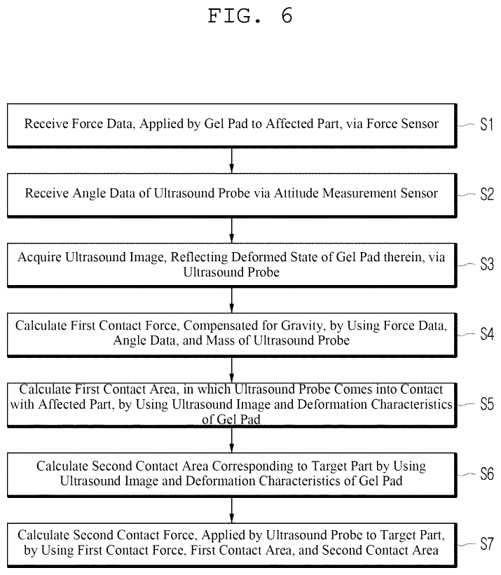

FIG. 6 is a flowchart illustrating a force measurement method for an ultrasonic diagnostic system according to one embodiment of the present invention.

Referring to FIG. 6, in the force measurement method for an ultrasonic diagnostic system, during ultrasound imaging using the ultrasound probe 200, the force sensor 111 measures the force applied to an affected part by the ultrasound probe 200 and provides force data to the control unit 130 in step S1, and the attitude measurement sensor 112 measures the angle formed between the height and vertical directions of the ultrasound probe 200 and provides angle data to the control unit 130 in step S2. In this case, the force data measured by the force sensor may be measured without considering whether a corresponding area is the contact portion with the affected part or a target part.

The control unit 130 generates and provides an ultrasound image via the ultrasound probe 200 in step S3. The ultrasound image reflects a deformation state in which the shape of the gel pad 210 is deformed in accordance with the curvature of the surface of the affected part as the gel pad 210 of the ultrasound probe 200 comes into contact with the affected part. Meanwhile, steps S1, S2, and S3 may be performed in parallel and simultaneously.

The control unit 130 compensates for the force applied to the affected part by the mass of the ultrasound probe 200 in the force data. To this end, the control unit 130 calculates the first contact force applied to the affected part by the ultrasound probe 200 by compensating for gravity by using the force data, the angle data, and the mass of the ultrasound probe in step S4.

The control unit 130 calculates the first contact area where the gel pad 210 of the ultrasound probe 200 comes into contact with the affected part based on the deformation state of the gel pad 210 reflected in the ultrasound image, and calculates the second contact area corresponding to the target part of the first contact areas in steps S5 and S6. The control unit 130 may use an image processing algorithm to calculate the first contact area and the second contact area from the ultrasound image.

The control unit 130 calculates the second contact force, applied by the ultrasound probe 200 to the target area of the affected part to be observed, by using the first contact force, the first contact area, and the second contact area in step S7.

After step S7, the control unit 130 may output the first contact force and second contact force, applied to the affected part by the ultrasound probe 200, along with the ultrasound image. Accordingly, it may be possible to know not only the global force applied to the affected part by the ultrasound probe 200 but also the local force applied to the target part by the ultrasound probe 200, so that considerably precise reading via the ultrasound image is enabled.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as unitary may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

1. An ultrasound diagnosis system, comprising:
a probe module including:
a force sensor configured to provide force data obtained by measuring a force applied by an ultrasound probe to an affected part;
an attitude measurement sensor configured to provide angle data obtained by measuring an angle formed between height and vertical directions of the ultrasound probe; and
a gel pad provided at an end of the ultrasound probe and configured to come into contact with the affected part; and
a control unit configured to calculate a first contact force applied by the ultrasound probe to the affected part by using an ultrasound image of the affected part acquired via the probe module, the force data, and the angle data, and to calculate a second contact force applied by the ultrasound probe to a target area of the affected part to be observed based on the first contact force.

2. The ultrasound diagnosis system of claim 1, wherein the gel pad comprises an elastic member whose shape is deformed in accordance with a curvature of a surface of the affected part.

3. The ultrasound diagnosis system of claim 1, wherein the first contact force is calculated by compensating for a force applied to the affected part by a mass of the ultrasound probe based on the force data.

4. The ultrasound diagnosis system of claim 3, wherein the first contact force is calculated by adding the force data to a value determined by the mass of the ultrasound probe and the angle data.

5. The ultrasound diagnosis system of claim 1, wherein:
the ultrasound image reflects therein a deformed state that occurs as the gel pad comes into contact with the affected part; and
the control unit extracts starting and ending points at which the gel pad comes into contact with the affected part from the ultrasound image and calculates a first contact area in which the gel pad comes into contact with the affected part, and extracts starting and ending points of the target part and calculates a second contact area in which the gel pad comes into contact with the target part.

6. The ultrasound diagnosis system of claim 5, wherein the second contact force is calculated as a force applied to the second contact area based on the first contact force and the first contact area.

7. The ultrasound diagnosis system of claim 6, wherein the second contact force is calculated using the first contact force, a thickness deformation amount of a local area for the target part, and a thickness deformation amount of an overall area in which the gel pad comes into contact with the affected part.

8. The ultrasound diagnosis system of claim 7, wherein the second contact force is calculated based on Equation 1 by considering a thickness of the gel pad and a thickness of the gel pad corresponding to any x coordinate:
$${F}_{target} = {F}_{probe} \frac{{\int }_{{x}_{1}}^{{x}_{2}} ( t - y ) dx}{{\int }_{{x}_{0}}^{{x}_{3}} ( t - y ) dx} \qquad ( 1 )$$
F_{target}: the second contact force
t: the thickness of the gel pad
y: the thickness of the gel pad corresponding to any x coordinate
x₀ and x₃: starting and ending points at which the gel pad comes into contact with the affected part x₁ and x₂: starting and ending points of the target part

9. A force measurement method for an ultrasound diagnosis system, the force measurement method being performed by an ultrasound diagnosis system including an ultrasound probe, the force measurement method comprising:
as an ultrasound image of an affected part is acquired via an ultrasound probe, measuring force data applied by the ultrasound probe to the affected part and angle data formed by height and vertical directions of the ultrasound probe; and
calculating a first contact force applied by the ultrasound probe to the affected part and a second contact force applied by the ultrasound probe to a target area of the affected part to be observed by using the ultrasound image, the force data, and the angle data.

10. The force measurement method of claim 9, wherein calculating the first contact force further comprises calculating the first contact force by compensating for a force with which the affected part is pressed by a mass of the ultrasound probe based on the force data.

11. The force measurement method of claim 9, wherein calculating the first contact force further comprises calculating the first contact force by using force data measured by a force sensor of the ultrasound probe, angle data measured by an attitude measurement sensor of the ultrasound probe, a mass of the ultrasound probe, and gravitational acceleration.

12. The force measurement method of claim 11, wherein calculating the first contact force comprises calculating the first contact force by compensating the force data for a value determined by the mass of the ultrasound probe and the angle data.

13. The force measurement method of claim 9, wherein calculating the second contact force further comprises:
extracting starting and ending points, at which a gel pad comes into contact with the affected part, from the ultrasound image reflecting therein a deformed state that occurs as the gel pad provided at an end of the ultrasound probe comes into contact with the affected part, and calculating a first contact area in which the gel pad comes into contact with the affected part; and
extracting starting and ending points of the target part from the ultrasound image, and calculating a second contact area in which the gel pad comes into contact with the target part.

14. The force measurement method of claim 13, wherein calculating the second contact force further comprises calculating the second contact force applied to the second contact area based on the first contact force and the first contact area.

15. The force measurement method of claim 14, wherein calculating the second contact force comprises calculating the second contact force by using the first contact force, a thickness deformation amount of a local area for the target part, and a thickness deformation amount of an overall area in which the gel pad comes into contact with the affected part.

16. The force measurement method of claim 15, wherein calculating the second contact force further comprises calculating the second contact force based on Equation 1 below by considering a thickness of the gel pad provided at the end of the ultrasound probe and configured to come into contact with the affected part and a thickness of the gel pad corresponding to any x coordinate:
$${F}_{target} = {F}_{probe} \frac{{\int }_{{x}_{1}}^{{x}_{2}} ( t - y ) dx}{{\int }_{{x}_{0}}^{{x}_{3}} ( t - y ) dx} \qquad ( 1 )$$
F_{target}: the second contact force
t: the thickness of the gel pad
y: the thickness of the gel pad corresponding to any x coordinate
x₀ and x₃: starting and ending points at which the gel pad comes into contact with the affected part
x₁ and x₂: starting and ending points of the target part
